(19) United States
(12) Patent Application Publication
(10) Pub. No.: US 2009/0209613 A1
(43) Pub. Date: Aug. 20, 2009

(54) Use of allymercaptocaptopril for treating or preventing obesity and obesity related diseases

(75) Inventors: David Mirelman, Ramat-Efal (IL); Aharon Rabinkov, Rechovot (IL); Talia Miron, Kfar-Haim (IL); Meir Wilchek, Rechovot (IL); Talma Rosenthal, Tel-Aviv (IL)

Correspondence Address: MARTIN D. MOYNIHAN d/b/a PRTSI, INC., P.O. BOX 16446, ARLINGTON, VA, 22215, US

(73) Assignee: Yeda Researc And Development Co. Ltd. at the Weizmann Institute of Science, Rechovot (IL)

(21) Appl. No.: 11/921,668

(22) PCT Filed: Jun. 13, 2006
(86) PCT No.: PCT/IL2006/000682

§ 371 Date: Dec. 6, 2007

Related U.S. Application Data
(60) Provisional application No. 60/689,558, filed on Jun. 13, 2005.

(51) Int. Cl. A61K 31/401; A61P 3/04

(52) U.S. Cl. 514/423

(57) ABSTRACT

Use of allylmercaptocaptopril for treating or preventing obesity and/or an obesity related disease or disorder is disclosed.

### FIELD AND BACKGROUND OF THE INVENTION

The present invention relates to pharmacology and, more particularly, to novel methods of treating or preventing obesity and/or obesity related diseases.

Obesity is a chronic, complex, multi-factorial disease, involving social, cultural, genetic, physiological and psychological components, and is associated with substantially increased morbidity and mortality. Over-nutrition is attributed as the cause of about 400,000 deaths a year in the USA, and may considered to be an epidemic. Based on the body-mass index, defined as the ratio of weight and squared height, (ranging normally from 18.5 to 24.9), about one third of the adult population is overweight (an index of from 25 to 29.9), and more than one quarter is obese (index greater than 30) (National Center for Health Statistics, 2000). Environmental and behavioral changes brought about by economic development and modernization have been linked to the rise in global obesity. The environmental factors which foster the tendency toward obesity include lack of physical activity combined with high-calorie, low-cost foods. The prevalence of overweight and obesity is increasing worldwide at an alarming rate in both developing and developed countries, in children and adults, men and women. The number of overweight and obese people has continued to increase since 1960, a trend that is not slowing down. Today, 64.5% of adult Americans—about 127 million—are categorized as being overweight or obese and nearly one-third (30.5%)—about 60 million—are obese, as reported in the National Health and Nutrition Examination Survey (NHANES) by the Centers for Disease Control and Prevention (CDC).

Obesity significantly increases the risk of illness from about thirty serious medical conditions and is associated with increases in deaths from all-causes. Among these are atherosclerosis, stroke, pancreatitis, myocardial infraction, hypertension, Type II or non-insulin dependent diabetes mellitus, hypercholestrosaemia, hyperlipidemia, metabolic syndrome, osteoarthritis, gallbladder disease and cancer of the breast, prostate and colon (National Task Force on the Prevention and Treatment of Obesity, 2000). Furthermore, each year, obesity causes at least 300,000 excess deaths in the U.S., being the second leading cause of unnecessary deaths. Healthcare costs of American adults with obesity amount to approximately 100 billion dollars.

There are several different treatment options for obesity, including: dietary therapy, physical activity, behavior therapy, drug therapy and surgery. For the majority of overweight and obese people, who find they are unable to change their lifestyle, drug therapy is the most favorable and applicable option. Although hundreds of millions of people are seeking drug therapy for the treatment of obesity, current drug therapies do not meet this need due to their undesired side effects and limited efficacy.

Medications for the treatment of obesity are currently approved for use in adults with a body-mass index of 30 or higher, or with a body-mass index of 27 or higher who have obesity-related medical problems (Physicians' Desk Reference, 2001). Approximately 10 percents of women and 3 percents of men with a body-mass index of 30 or higher reportedly use weight-loss medications.

Exemplary treatment approaches and agents are disclosed in U.S. Pat. Nos. 3,867,539 (by internally administering Histidine); 4,446,138 (by administering L-Dopa and Molindone); 4,588,724 (by delivering β adrenergic stimulator or α-2 adrenergic inhibitor); 4,745,122 (by administering paroxetine); 5,019,594 (by administering an indirect-acting sympathomimetic drug and tyrosine or a tyrosine precursor); 5,300,298 (by administering certain substituted 8-phenylxanthines); 5,403,851 (by administering substituted tryptamine and phenalkylamine and related compounds); 5,567,714 (by inhibiting physiological conditions associated with an excess of neuropeptide Y); 5,573,774 (by administering nicotine metabolites); 5,578,613 (by administering certain 2-phenyl-3-aroylbenzothiophenes (benzothiophenes)); and 5,900,411.

Although some prior art treatment approaches are effective in controlling obesity over a relatively short time period, unfortunately, such approaches have not been very successful in a long-term treatment of this disorder.

Thus a widely recognized need for, and it would be highly advantageous to have, novel and efficacious methods for treating obesity or related diseases.

Allylmercaptocaptopril (CPSSA), represented by Formula I below, is a recently disclosed conjugate of allicin, a biologically and therapeutically active compound derived from garlic, and of captopril, an angiotensin-converting enzyme (ACE)-inhibiting compound.

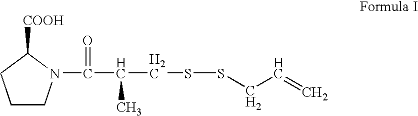

The preparation and beneficial therapeutic effects of CPSSA are described in WO 02/096871 and by Miron et al., in Amer. J Hypertension, 2004, 17, 71-73, which are incorporated by reference as if fully set forth herein. CPSSA has been shown to combine the advantages of the ACE-inhibiting captopril with the beneficial effects of allicin, while circumventing the limitations associated with each of these components when utilized alone. For example, WO 02/096871 and Miron et al. (supra) CPSSA reacts very sluggishly with serum proteins, and is thus stable in blood or plasma of mammals.

The effect of CPSSA as a weight management agent, however, have not been demonstrated nor suggested in these publications.

### SUMMARY OF THE INVENTION

The present inventors have surprisingly found that orally-administered CPSSA efficiently reduced weight gain in obese rats.

Thus, according to one aspect of the present invention there is provided a method of treating or preventing obesity and/or an obesity related disease or disorder. The method is effected by administering to a subject in need thereof a therapeutically effective amount of allylmercaptocaptopril (CPSSA), thereby treating or preventing obesity or the obesity related disease in the subject.

According to another aspect of the present invention there is provided a use of CPSSA for producing a medicament for treating obesity and/or an obesity related disease or disorder.

According to yet another aspect of the present invention there is provided an article of manufacture which includes packaging material and a pharmaceutical composition identified for use in treating or preventing obesity and/or an obesity related disease or disorder of a subject being contained within the packaging material, the pharmaceutical composition including, as an active ingredient, CPSSA and a pharmaceutically acceptable carrier.

According to further features in preferred embodiments of the invention described below, the subject is a human.

According to still further features in the described preferred embodiments the obesity related disease is selected from the group consisting of atherosclerosis, stroke, myocardial infraction, hypertension, Type II or non-insulin dependent diabetes mellitus, hypercholestrosaemia, hyperlipidemia, pancreatitis, metabolic syndrome, osteoarthritis, gallbladder disease and cancer.

According to still further features in the described preferred embodiments the obesity related disease is metabolic syndrome.

According to still further features in the described preferred embodiments the administering is effected orally.

The present invention successfully addresses the shortcomings of the presently known configurations by providing highly efficient methods and articles of manufacturing for treating obesity or obesity related diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein the term “about” refers to ±10%.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In each of the methods described herein, the CPSSA is preferably not used for treating patients also having a medical condition that has previously been described as treatable by CPSSA.

Thus, in each of the various aspects of the present invention, the subjects treated by any of the methods described herein are preferably subjects that do not also suffer from a condition that has already been described in the art as treatable by CPSSA, as described herein. Such conditions include, for example, any of the conditions described in WO 02/096871 and Miron et al. (supra).

### BRIEF DESCRIPTION OF THE DRAWINGS

The invention is herein described, by way of example only, with reference to the accompanying drawings. With specific reference to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents comparative plots demonstrating the average body weight gain over time of SHROB model rats which have been fed with standard rat chow supplemented with allylmercaptocaptopril (CPSSA, denoted as BL-2040, yellow stars), captopril (magenta squares) or non-supplemented chow (control, blue diamonds);

FIG. 2 presents comparative plots demonstrating the average food consumption by SHROB model rats which were given ad libitum access to standard rat chow supplemented with allylmercaptocaptopril (CPSSA, denoted as BL-2040, yellow stars), captopril (magenta squares) or non-supplemented chow (control, blue diamonds); and

FIG. 3 is a bar graph presenting the average systolic blood pressure measured in SHROB model rats which were given ad libitum access to standard rat chow supplemented with allylmercaptocaptopril (CPSSA, turquoise bars), captopril (light blue bars) or non-supplemented chow (control, dark blue bars).

### DESCRIPTION OF THE PREFERRED EMBODIMENTS

The present invention is of novel methods of treating or preventing obesity and related diseases via administering allylmercaptocaptopril (CPSSA) to a subject. The present invention is further of uses of CPSSA in the manufacture of medicaments for treating obesity and obesity related diseases and of articles-of-manufacturing containing same.

The principles and operation of the methods, uses and articles of manufacture according to the present invention, may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As described hereinabove, obesity and obesity-related diseases, such as atherosclerosis, stroke, pancreatitis, myocardial infraction, hypertension, Type II diabetes, hypercholestrosaemia, hyperlipidemia, metabolic syndrome and cancer (e.g., breast, prostate and colon), are a major concern in the modern western world. Over the years a limited number of treatment approaches have been suggested, but none was effective in controlling obesity over in the long-term.

Allylmercaptocaptopril (CPSSA) has been shown to have various beneficial therapeutic effects (WO 02/096871 and Miron et al., supra). In particular, it has been shown that CPSSA significantly decreases blood pressure and reduces the serum levels of triglycerides and insulin. CPSSA is further stable in blood or plasma of mammals, and thus, low doses thereof are sufficient to achieve effectiveness in mammals.

So far, none of the studies on CPSSA has shown or suggested any correlation between CPSSA and body weight management.

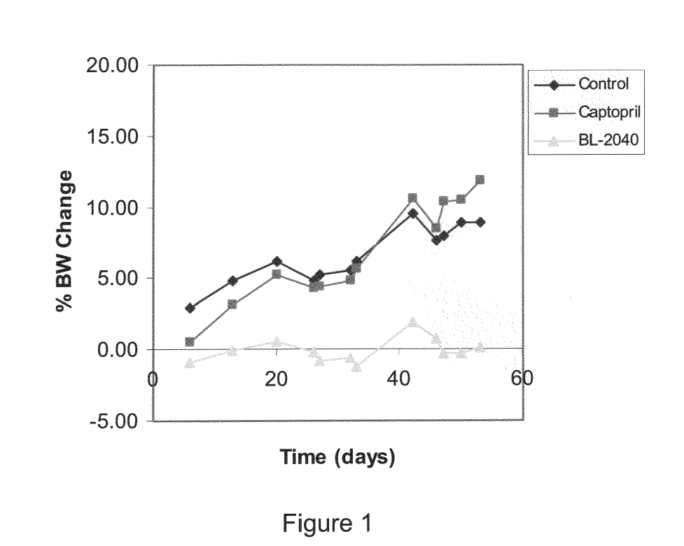

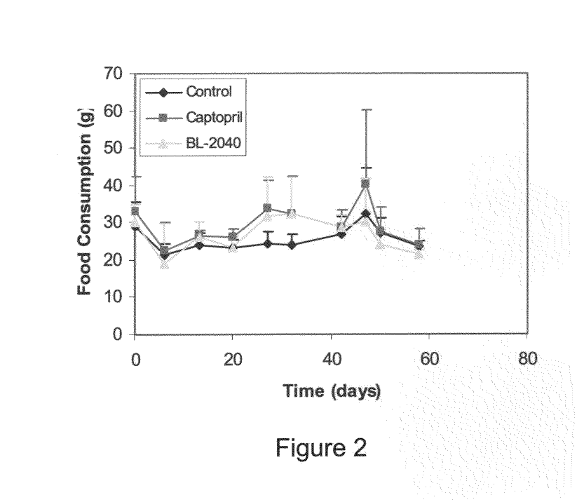

The present inventors have now unexpectedly uncovered that CPSSA can effectively prevent body weight gain in model animals. As is demonstrated in the Examples section that follows, while no differences were observed in the food uptake in CPSSA-treated, captopril-treated and non-treated model rats throughout the experiment (FIG. 2), a substantial decrease in body weight gain was observed in CPSSA-treated obese model rats (FIG. 1), as compared to non-treated and captopril-treated rats. As is further demonstrated in the Examples section that follows, the beneficial effect of CPSSA was maintained over a long period of time (over 50 days) and was also accompanied by a decrease in the systolic blood pressure of the treated model animals (a known effect of one of the CPSSA parent compounds, captopril).

Thus, according to one aspect of the present invention, there is provided a method of treating or preventing obesity and/or an obesity related disease or disorder in a subject. The method is effected by administering to a subject in need thereof a therapeutically effective amount of allylmercaptocaptopril (CPSSA), so as to treat or prevent obesity or the obesity related disease in the subject.

As used herein the term “subject” refers to a mammal, and is preferably a human. A “subject in need thereof” is a subject suffering or being predisposed to obesity or obesity related diseases. The phrase “subject in need thereof” preferably refers to subjects suffering or being predisposed to obesity or obesity related diseases or disorders that do not also suffer from a condition that has already been described in the art as treatable by CPSSA, as described herein. Such conditions include, for example, hypertension, hyperlipidemia, and diabetes, all being independent of (not related to) obesity, as described in WO 02/096871 and Miron et al. (supra).

The phrases “treating or preventing” and “treatment or prevention” used herein encompass the complete range of therapeutically positive effects of administrating CPSSA to a subject including reduction of, alleviation of, and relief from obesity or obesity related diseases or disorders. Treatment or prevention includes the prevention or postponement of development of obesity or obesity related diseases or disorders, prevention or postponement of development of symptoms and/or a reduction in the severity of such symptoms that will or are expected to develop. These further include ameliorating existing symptoms, preventing additional symptoms and ameliorating or preventing the underlying metabolic causes of symptoms.

The term “obesity” as used herein refers to an excess of body weight in a subject. As per the American Obesity Association an adult human having a Body Mass Index (MBI) over 25 is considered to have an excess of body weight. An excess of body weight in turn may considerably increase the risk of developing, or enhance progression of a number of diseases, which are referred to herein as “obesity related diseases or disorders”.

The term “therapeutically effective amount” or “pharmaceutically effective amount” denotes that dose of an active ingredient or a composition comprising the active ingredient that will provide the therapeutic effect for which the active ingredient is indicated, herein, for example, treating obesity and an obesity related disease or disorder.

For any of the compounds utilized in this and other aspects of the invention, a therapeutically effective amount, also referred to herein interchangeable as a therapeutically effective dose, can be estimated initially from cell culture assays. Such information can be used to more accurately determine useful doses in humans. Initial dosages can also be estimated from in vivo data. Using these initial guidelines one having ordinary skill in the art could determine an effective dosage in humans.

Moreover, toxicity and therapeutic efficacy of the compounds described herein can be determined by standard pharmaceutical procedures in cell cultures or experimental animals. Compounds which exhibit high therapeutic indices are preferred. The data obtained from these cell cultures assays and animal studies can be used in formulating a dosage range that is not toxic for use in human. The exact formulation, route of administration and dosage can be chosen by the individual physician in view of the patient's condition. (See, e.g., Fingl et al., 1975, In: The Pharmacological Basis of Therapeutics, chapter 1, page 1).

As used herein, the term “administering” refers to a method for bringing a compound, herein the CPSSA, and an affected area together in such a manner that the compound can affect the affected area.

In a preferred embodiment, the CPSSA is conveniently administered orally.

As used herein, the term allylmercaptocaptopril, abbreviated herein as CPSSA, encompasses CPSSA itself, as presented in Formula I above, as well as derivatives and analogs thereof, such as described, for example, in WO 02/096871.

As used herein, the term “derivative” describes a compound (herein CPSSA), which has been subjected to a chemical modification (e.g., substitution, reduction, oxidation and the like), while maintaining most of its structural features.

The term “analog” describes a compound that is structurally related to the parent compound (herein CPSSA) and hence typically exhibits the same performance as the parent compound.

Since, as discussed hereinabove, obesity is oftentimes implicated in other conditions, the present invention can also be utilized to treat or prevent diseases and disorders which are associated with obesity.

For example, obesity characterized by excess body weight greater than 30% doubles the incidence of coronary diseases in subjects under 50 years of age; an excess body weight of 20% doubles the risk of high blood pressure; an excess body weight of 30% triples the risk of developing non-insulin dependent diabetes; and an excess body weight of 30% multiplies the risk of developing hyperlipidemia by six folds.

Thus, examples of obesity related diseases and disorders include, but are not limited to, atherosclerosis, stroke, myocardial infraction, hypertension, Type II or non-insulin dependent diabetes mellitus, hypercholestrosaemia, hyperlipidemia, pancreatitis, metabolic syndrome, osteoarthritis, gallbladder disease and cancer (e.g., breast, prostate and colon).

As used herein, the term “atherosclerosis” refers to a condition characterized by irregularly distributed deposits of lipid and lipoprotein in the intima of large and medium-sized arteries often covered with a fibrous cap and calcification.

The term “myocardial infarction” refers to a sudden insufficiency of the blood supply to the heart, and the term “stroke” refers to a sudden development of focal neurological deficits usually related to impaired cerebral blood supply (cerebrovascular stroke).

The term “hyperlipidemia” as used herein, refers to a condition in which a patient exhibits elevated concentrations of any or all lipids in plasma.

The term “hypercholesterolemia” refers to an abnormally large amount of cholesterol present in the cells and/or plasma of circulating blood.

The term “pancreatitis” refers to inflammation of the pancreas.

The term “Type II or non-insulin dependent diabetes” refers to a type of diabetes in which insulin production is normal while insulin resistance is developed.

According to the American diabetic association, an adult person has metabolic syndrome if he has 3 of the following 5 indications:

(i) BMI over 25 or if his waist circumference is 102 cm (in Europe ≧94) for men and ≧88 cm (in Europe ≧80) for woman;

(ii) Blood triglycerides level that is equal to or higher than 150 mg/dl;

(iii) Blood HDL cholesterol level lower than 40 mg/dl (for men) or lower than 50 mg/dl (for women);

(iv) Blood glucose level that is equal to or higher than 100 mg/dl; and/or

(v) Blood pressure that is equal to or higher than 130/85 mmHg.

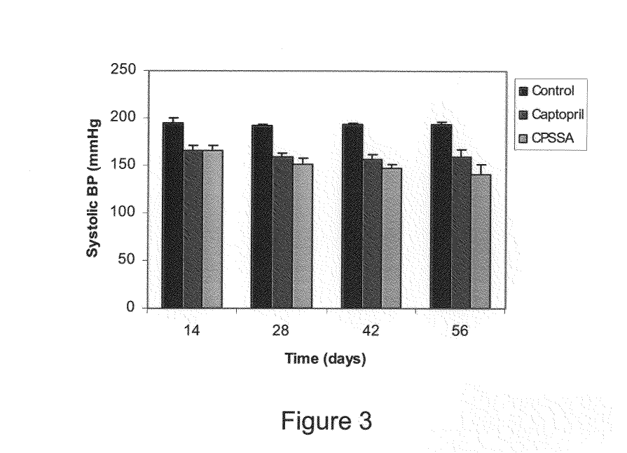

As illustrated in the Examples section that follows, administering CPSSA to model obese animals substantially reduce their body weight gain (FIG. 1) and further beneficially affected (lowered) their blood pressure (FIG. 3). As taught in WO 02/096871, CPSSA is also effective in reducing blood triglycerides and insulin levels, in addition to its effect in reducing blood pressure. Taken together the teachings of WO 02/096871 and the surprising findings described herein, CPSSA can be utilized for treating or preventing at least four of the five indications associated with metabolic syndrome, and can thus be efficiently used for treating or preventing a metabolic syndrome in a subject.

Thus, CPSSA can be further utilized in the manufacture of a medicament for treating or preventing obesity, for treating or preventing an obesity related disease or disorder, as described herein, and particularly, for treating or preventing a metabolic syndrome.

In each of the aspects of the present invention, the CPSSA can be utilized in combination with an additional active agent that is beneficial for the treatment of the treated condition, namely, obesity and/or an obesity related disease or disorder.

Such additional active ingredients can include, for example, appetite suppressants (for treating obesity), vasodilators (for treating hypertension), cholesterol absorption inhibitors and cholesterol generation inhibitors (for treating hypercholesterolemia), fat absorption inhibitors and fat generation inhibitors (for treating obesity, hyperlipidemia, poorly controlled diabetes, atherosclerosis and pancreatitis), anti-diabetic agents and the like. These additional active ingredients may act synergistically or additively with the CPSSA, thereby increasing the overall efficacy thereof.

Representative examples of appetite suppressants include, without limitation, fenfluramine, dexfenfluramine, phentiramine, sulbitramine, orlistat, pyrazolecarboxamide, neuropeptide Y5 receptor antagonists, leptin, β-3-adrenergic receptor agonists, serotonin agonists and PPARγ antagonists.

Representative examples of vasodilators include, without limitation, diuretics, such as, but not limited to, hydrochlorothiazide and indapamide, beta-blockers, such as, but not limited to, atenolol, labetalol hydrochloride and pindolol; central alpha-2 agonists, such as, but not limited to, clonidine hydrochloride and methyldopa; alpha-1-receptor blockers, such as, but not limited to, doxazosin mesylate; ACE Inhibitors, such as, but not limited to, benazepril hydrochloride and captopril; angiotensin-II-receptor blockers, such as, but not limited to, losartan potassium; calcium channel blockers, such as, but not limited to, diltiazem CD/SR/XR, nifedipine XL and verapamil hydrochloride; direct vasodilators, such as hydralazine hydrochloride and minoxidil, and peripheral alpha-adrenergic antagonists, such as guanethidine monosulfate and reserpine.

Representative examples of agents for the treatment of atherosclerosis include, without limitation, angiotensin-converting enzyme inhibitors (such as captopril, enalapril, or lisinopril); antiarrhythmic drugs (such as amiodarone); anticoagulants, antiplatelets or thrombolytics (such as aspirin); centrally acting drugs (such as clonidine, guanfacine or methyldopa); digitalis drugs (such as digoxin); diuretics (such as chlorthalidone); nitrates (such as nitroglycerin); peripheral adrenergic antagonists (such as reserpine); and vasodilators (such as hydralazine).

Representative examples of agents for the treatment of diabetes include, without limitation, a meglitinide (such as repaglinide or nateglinide), a biguanide (such as metformin), a thiazolidinedione (such as rosiglitazone, troglitazone or pioglitazone), and an alpha-glucosidase inhibitor (such as acarbose or meglitol) and insulin.

Non-limiting examples of agents for the treatment of dyslipidemia include fibrates, HMG-CoA reductase inhibitors, bile acid sequestrants, cholesterol absorption inhibitors, cholesterol biosynthesis inhibitors, nicotinic acid and derivatives thereof.

HMG-CoA reductase inhibitors (statins) are well known drugs that effectively reduce LDL-cholesterol levels by inhibiting the enzyme that regulates the rate of cholesterol production and increasing the clearance of LDL-cholesterol present in the blood by the liver. Representative examples of commonly prescribed statins include Atorvastatin, Fluvastatin, Lovastatin, Pravastatin and Simvastatin.

Proliferative Activated Receptor (PPAR) agonists, also known as fibrates, are fatty acid-activated members of the nuclear receptor superfamily that play important roles in lipid and glucose metabolism, and have been implicated in obesity-related metabolic diseases such as hyperlipidemia, insulin resistance (diabetes), and coronary artery disease. Fibrates are generally effective in lowering elevated plasma triglycerides and cholesterol and act as PPAR agonists. The most pronounced effect of fibrates includes a decrease in plasma triglyceride-rich lipoproteins (TRLs). Levels of LDL cholesterol (LDL-C) generally decrease in individuals with elevated baseline plasma concentrations, and HDL cholesterol (HDL-C) levels are usually increased when baseline plasma concentrations are low. Non-limiting examples of commonly prescribed fibrates include bezafibrate, gemfibrozil and fenofibrate.

Representative examples of cholesterol absorption inhibitors include ezetimibe. Ezetimibe is the first of a new class of cholesterol absorption inhibitors that potently and selectively inhibits dietary and biliary cholesterol absorption at the brush border of the intestinal epithelium, without affecting the absorption of triglyceride or fat-soluble vitamins. Ezetimibe thus reduces overall cholesterol delivery to the liver, secondarily inducing increased expression of LDL receptors, resulting in an increased removal of LDL-C from the plasma.

Cholesterol absorption may also be affected by Cholesteryl Ester Transfer Protein (CETP) inhibitors, which play a major role in atherogenesis, by reducing cholesteryl ester accumulation within macrophages and the arterial wall, and thus reducing foam cell formation and affecting the cholesterol absorption. The most promising presently known CETP inhibitor is avisimibe.

Representative examples of cholesterol biosynthesis inhibitors include squalene inhibitors (such as monooxygenase and synthase). Squalene is an isoprenoid compound structurally similar to beta-carotene, and is an intermediate metabolite in the synthesis of cholesterol. In humans, about 60 percent of dietary squalene is absorbed. It is transported in serum generally in association with very low density lipoproteins and is distributed ubiquitously in human tissues, with the greatest concentration in the skin, where it is one of the major components of skin surface lipids. Squalene inhibitors (e.g., monooxygenase and synthase) serve as cholesterol biosynthesis inhibitors.

Nicotinic acid is a known agent that lowers total cholesterol, LDL-cholesterol, and triglyceride levels, while raising HDL-cholesterol levels. There are three types of nicotinic acid drugs: immediate release, timed release, and extended release. Nicotinic acid or niacin, the water-soluble B vitamin, improves all lipoproteins when given in doses well above the vitamin requirement.

In each of the aspects of the present invention, CPSSA can be utilized either per se or as a part (active ingredient) of a pharmaceutical composition.

As used herein a “pharmaceutical composition” refers to a preparation of one or more of the active ingredients or agents described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Although not presently preferred, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g., allylmercaptocaptopril) effective to prevent, alleviate or ameliorate symptoms of a disorder (e.g., an excessive overweight) of the subject being treated.

Determination of a therapeutically effective amount is well within the capability of those skilled in the art, especially in light of the detailed disclosure provided herein. Preferably, a therapeutically effective amount ranges between 20 mg and 300 mg as a total daily dose for human subjects.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to levels of the active ingredient are sufficient to substantially affect the body weight gain of an individual. Dosages necessary to achieve the desired effect will depend on individual characteristics and route of administration. Detection assays can be used to determine plasma concentrations.

Depending on the severity and responsiveness of the condition to be treated, dosing can be of a single dose, or preferably two or more daily doses, with course of treatment lasting from several days to several weeks or until diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack.

Pharmaceutical compositions according to the present invention may further include one or more additional active ingredients which may affect the condition being treated.

The pharmaceutical compositions may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as detailed herein.

Thus, according to an embodiment of the present invention, there is provided an article of manufacture which comprises the pharmaceutical composition, as described herein, packaged in a packaging material and identified in print, in or on the packaging material, for use in the treatment of a medical condition in which treatment with CPSSA is beneficial. As discussed in detail hereinabove, such conditions include, for example, obesity and obesity related diseases, as described herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Effect of CPSSA on Reducing Body Weight Gain

### Material and Methods:

Animals: Obese spontaneously hypersensitive rats with a naturally occurring knockout of the leptin receptor (SHROB; Koletsky et al., pp. 143-158 in: Sima AAF and Shafrir (eds) Animal Models of Diabetes: A Primer, Harwood, Singapore, 2001) were used. Adult (4-5 months old) male and female SHROB were maintained essentially as described by Velliquette and Emsberger (JPET 307: 1104-1111, 2003). Briefly, animals were housed individually at a constant temperature of 21° C. on a 12:12 hours light/dark cycle. The animals were given ad libitum access to food (Teklad 8664 rat chow) and were maintained for a period of 60 days. The procedures were carried out in accordance with applicable laws and accepted guidelines for animal welfare.

Drugs: Allylmercaptocaptopril (CPSSA) was produced using the procedure described in WO 02/096871. Captopril, a commercial drug used for treating hypertension, was purchased from SIGMA (St. Louis, Mo., USA).

### Activity Assays:

Either CPSSA or captopril were dissolved in a 20% ethanol solution, chilled on ice, and were then thoroughly mixed with powdered rat chow in equivalent molar amounts of 1.107 and 0.827 gram/kg, respectively.

Eight SHROB rats (4 males, 4 females) were used in each treatment group. Animals were fed with chow supplemented with either CPSSA or captopril, or with standard (non-supplemented) chow diet, which served as a control. Measurements of body weight, food consumption and blood pressure were conducted 3 times a week for the duration of the experiment.

### Experimental Results:

The average body weight gain over time in rats fed with chow supplemented is with either CPSSA or captopril, or with non-supplemented chow, are presented in FIG. 1.

As can be seen in FIG. 1, captopril had no effect on body weight gain, as compared with the non-treated control. In sharp contrast, CPSSA effectively prevented body weight gain. For example, on day 20, the average body weight gain of the control, captopril-treated and CPSSA-treated rats were 6.2, 7.3 and 0.6%, respectively. Similarly, on day 53, the average body weight gain of the control, captopril-treated and CPSSA-treated rats were 8.95, 11.8 and −0.1%, respectively.

The average food consumption by rats which were given ad libitum access to rat chow supplemented with either CPSSA or captopril, or with non-supplemented chow is presented in FIG. 2, and show that no differences were observed in the food uptake within the various treatment groups throughout the experiment.

In addition to preventing body weight gain, CPSSA substantially reduced the blood pressure of treated animals. As can be seen in FIG. 3, the average systolic blood pressure of animals treated with CPSSA was 165.6, 146.8 and 140.3, on days 14, 28 and 56, respectively. In comparison, the average systolic blood pressure of non-treated control animals was 195.2, 192.0 and 193.0, on days 14, 28 and 56, respectively. The average systolic blood pressure of animals treated with captopril was 165.5, 159.3 and 159.7, on days 14, 28 and 56, respectively.

These results clearly indicate the capacity of orally administered CPSSA to effectively reduce or even prevent body weight gain in animals, in addition to being highly effective in lowering their blood pressure.

1. A method of treating or preventing obesity and/or an obesity related disease or disorder, the method comprising administering to a subject in need thereof a therapeutically effective amount of allylmercaptocaptopril (CPSSA), thereby treating or preventing obesity or the obesity related disease in said subject.

2. The method of claim 1, wherein said subject is a human.

3. The method of claim 1, wherein said obesity related disease or disorder is selected from the group consisting of atherosclerosis, stroke, pancreatitis, myocardial infraction, hypertension, Type II or non-insulin dependent diabetes mellitus, hypercholestrosaemia, hyperlipidemia, metabolic syndrome, osteoarthritis, gallbladder disease and cancer.

4. The method of claim 3, wherein said obesity related disease or disorder is a metabolic syndrome.

5. The method of claim 1, wherein said administering is effected orally.

6. The method of claim 1, further comprising administering to the subject a therapeutically effective amount of an additional active agent.

7. The method of claim 6, wherein said additional active agent is selected from the group consisting of an appetite suppressant, a vasodilator, a cholesterol absorption inhibitor, a cholesterol generation inhibitor, a fat absorption inhibitor, a fat generation inhibitor, an anti-diabetic agent, a chemotherapeutic agent and an anti-inflammatory agent.

8-12. (canceled)

13. An article of manufacture, comprising a packaging material and a pharmaceutical composition identified for use in treating or preventing obesity and/or an obesity related disease or disorder of a subject being contained within the packaging material, said pharmaceutical composition including, as an active ingredient, allymercaptocaptopril and a pharmaceutically acceptable carrier.

14. The article of manufacture of claim 13, wherein said obesity related disease or disorder is selected from the group consisting of atherosclerosis, stroke, pancreatitis, myocardial infraction, hypertension, Type II or non-insulin dependent diabetes mellitus, hypercholestrosaemia, hyperlipidemia, metabolic syndrome, osteoarthritis, gallbladder disease and cancer.

15. The article of manufacture of claim 14, wherein said obesity related disease or disorder is a metabolic syndrome.

16. The article of manufacture of claim 13, wherein said pharmaceutical composition further comprises an additional active agent.

17. The article of manufacture of claim 16, wherein said additional active agent is selected from the group consisting of an appetite suppressant, a vasodilator, a cholesterol absorption inhibitor, a cholesterol generation inhibitor, a fat absorption inhibitor, a fat generation inhibitor, an anti-diabetic agent, a chemotherapeutic agent and an anti-inflammatory agent.
